(19) United States
(12) Patent Application Publication
(10) Pub. No.: US 2003/0157653 A1
(43) Pub. Date: Aug. 21, 2003

(54) Method for producing monoglycosidated flavonoids

(76) Inventors: Hans Leonard Ohrem, Jugenheim (DE); Achim Schwammle, Darmstadt (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/203,537

(22) PCT Filed: Sep. 2, 2001
(86) PCT No.: PCT/EP01/01447

(51) Int. Cl. C12N 9/00; C12P 19/56

(52) U.S. Cl. 435/78; 435/183

(57) ABSTRACT

The present invention relates to a process for the production of monoglycosidated flavonoids by enzymatic hydrolysis of rutinosides, in which the enzymatic hydrolysis is carried out using an enzyme immobilized on a support. This process can avoid high enzyme costs whilst at the same time a high degree of automation, combined with high space-time yields is achieved.

The present invention relates to a process for the production of monoglycosidated flavonoids by enzymatic hydrolysis of rutinosides. During this operation the rhamnose radical of the rutinosides is enzymatically cleaved.

For the purposes of the present invention rutinosides are regarded as being compounds containing an aglycosuric component to which a radical of formula (I)

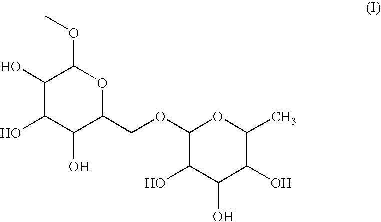

is linked through a glycosidic bond. For example, rutinosides are flavonoids containing the bisgylcosidic unit illustrated in formula (I). Rhamnose and/or the corresponding glucopyranosides can be obtained from the rutinosides. The glucopyranosides are derived from the rutinosides in that they contain, instead of the radical of formula (I), a radical of the formula (I*)

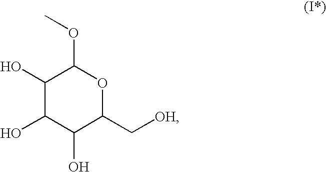

which is bonded to the aglycosuric component. For example, both rhamnose and isoquercetin can be obtained from rutin.

Rhamnose is a monosaccharide which occurs naturally in many places, but mostly in only small quantities. An important source of rhamnose comprises, for example, the glycosidic radicals of natural flavonoids, such as rutin, from which rhamnose can be obtained by elimination of glycoside. Rhamnose is significant, for example, as a starting material for the preparation of non-natural aroma substances, such as furaneol.

Isoquercetin is a monoglycosidated flavonoid of the following structural formula (II)

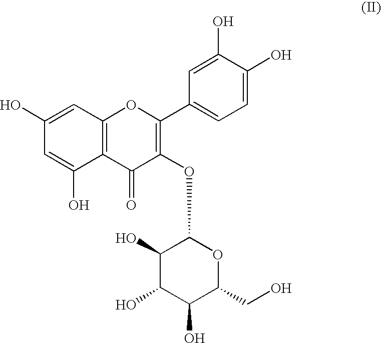

By flavonoids (latin: flavu=yellow), which are widespread dyes in plants, are meant, for example, glycosides of flavones, with which they have in common the parent structure of flavone (2-phenyl4H-1-benzopyranone-4).

The aglycosuric component of the flavonoids is the so-called aglycon. Isoquercetin is, for example, a glycoside of the aglycon quercetin (2-(3,4-dihydrophenyl)-3,5,7-trihydroxy-4H-1-benzopyranone-4), which differs from flavone by the presence of five hydroxyl groups. In isoquercetin the carbohydrate radical glucose is bonded to the hydroxyl group in position 3 of quercetin. Isoquercetin is, for example, designated as quercetin-3-O-β-D-glucopyranoside or 2-(3,4-dihydroxyphenyl)-3-(β-D-glucopyranosyloxy)-5,7-dihydroxy-4H-1-benzopyranone-4. However it is also known, for example, under the tradename Hirsutrin.

Flavonoids or flavonoid blends are used, for example, in the food and cosmetics industries, where they are becoming increasingly significant. Particularly monoglycosidated flavonoids, such as isoquercetin, are characterized by good absorption in the human body.

An example of a naturally occurring flavonoid having a bisglycosidic unit is rutin, which has the following structural formula (III):

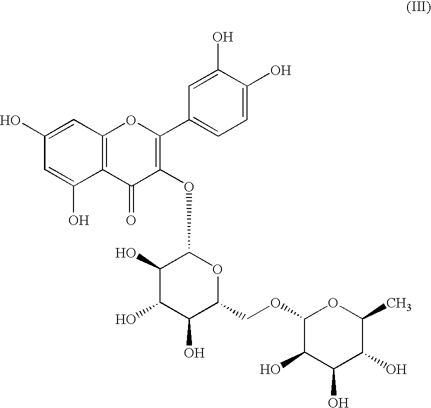

Rutin, like isoquercetin, is likewise a glycoside of the aglycon quercetin, the carbohydrate radical rutinose being linked to the hydroxyl group in position 3 of quercetin. The carbohydrate radical in the rutin comprises a glucose unit linked in positions 1 and 6 and a terminally bound rhamnose or 6-deoxymannose unit. Rutin is known, for example, as quercetin-3-O-β-D-rutinoside or 2-(3,4-dihydroxyphenyl)-3-{[6-O-(6-deoxy-α-mannopyranosol)-β-D-glucopyranosyl]oxy}-5,7-dihydroxy-4H-1-benzopyranone4. But it is also known, for example, by the names of sophorin, birutan, rutabion, taurutin, phytomelin, melin, or rutoside.

Rutin forms with three molecules of water of crystallization pale yellow to greenish needles. Anhydrous rutin has the property of a weak acid, turns brown at 125° C. and decomposes at 214-215° C. Rutin, which occurs in many plant species—frequently as a companion substance of vitamin C—, eg, in citrus species, in yellow pansies, species of forsythia and acacia, various solanum and nicotiana species, capers, lime blossom, St. John's wort, tea etc., was isolated in 1842 from Ruta graveolens. Rutin can also be obtained from leaves of the buckwheat and the Eastern Asian dyer's drug Wie-Fa (Sophora japonica, Fabaceae), which contains 13-27% of rutin.

It is desirable to prepare both rhamnose and monoglycosidated flavonoid from natural raw materials, for example, from flavonoids containing a bisglycosidic unit. In this context, for example, the cleavage of rutinosides to rhamnose and the corresponding glucopyranosides is interesting.

Enzymatically catalyzed preparations of rhamnose are disclosed in the literature. For example, EP-A 0,317,033 describes a process for the production of L-rhamnose, in which rhamnosidic bonding of glycosides containing rhamnose bonded in the terminal position, is achieved by enzymatic hydrolysis. This cleavage is carried out on the substrate usually present as a suspension in an aqueous medium. However, these reactions are mostly poorly selective. For example, the bisglycosidic structure of the carbohydrate radical in the rutin often leads to a mixture of the two monosaccharides glucose and rhamnose. Also, there are usually formed high portions of the aglycon quercetin and other undesirable by-products.

Furthermore, enzymatically catalyzed cleavages of rutin are also described in JP-A 0,121,3293. However, such reactions carried out in aqueous media are likewise usually poorly selective.

These processes described above use the enzyme in solution, ie as a native substance. The process involves the direct addition of the enzyme to the reaction solution. Although these processes can be carried out on a laboratory scale, they are not feasible for industrial use, since the enzyme cannot be regained from the reaction solution for reuse. However, to use these expensive enzymes only once is not economical on an industrial scale.

It is known that enzymes can be used industrially when they are bound to a support. This procedure is referred to as “immobilization”. The term “bound (or immobilized) enzymes” includes, according to The European Federation of Biotechnology (1983), all enzymes “ . . . which exist in a state allowing the reuse thereof” (Helmut Uhlig, Technische Enzyme and ihre Anwendung, Carl Hanser Verlag, Munich/Vienna 1991, pp 198). Despite this advantage, immobilization is not suitable for all enzymatic processes, however, and has hitherto been adopted to a limited extent only. In particular, only two bound enzymes are in use on a commercial-scale: immobilized glucose isomerase for glucose isomerization and immobilized penicillin amidase for penicillin-G cleavage. Often bound-enzyme processes cannot stand against free-enzyme or chemical processes. It frequently happens that the enzymes or the reaction conditions are not suitable for immobilization. Thus there exists no universal method of immobilization, and each enzyme must be considered individually.

For example, aqueous systems such as are important for the serviceability of enzymes give rise to solubility problems when rutinosides are used as substrate in enzymatic hydrolysis. This reaction is therefore preferably carried out using a supersaturated substrate solution, ie in the form of a rutinoside suspension. However, a supersaturated solution, in which the substrate is present as solid matter, precludes the use of immobilization methods. There is a lack of selectivity between the raw material, the product particles, and the bound enzyme.

It is thus an object of the present invention to provide a process for the production of monoglycosidated enzymes which can be used on an industrial scale with the avoidance of high enzyme costs whilst achieving a high degree of automation and high space-time yields and high productivity and selectivity.

This object is achieved by means of a process for the production of monoglycosidated flavonoids by enzymatic hydrolysis of rutinosides in which the enzyme used for the enzymatic hydrolysis is one which has been immobilized on a support.

We have found, surprisingly, that despite the slight solubility of rutinosides, enzymatic hydrolysis using a bound enzyme is possible. The immobilization makes it possible to carry out the process continuously or batchwise and with a high degree of efficiency compared with the reaction involving native enzyme. The process of the invention is particularly distinguished in that it makes possible a high degree of automation of the entire process including feedback of the solvent and monitoring of the enzyme activity.

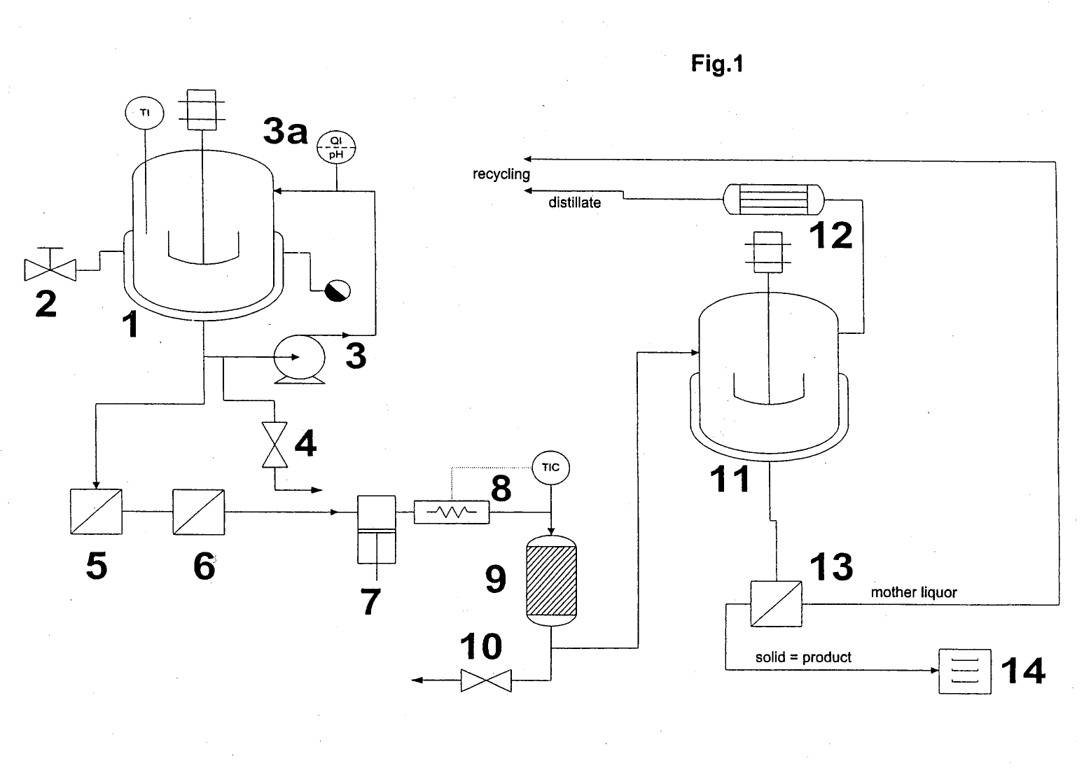

FIG. 1 illustrates the continuous production of isoquercetin from rutin as an example of the process of the invention.

Suitable rutinosides for use in the process of the invention are those containing, as aglycosuric component or aglycon, a parent substance comprising 2-phenyl4H-1-benzopyranone-4 which carries a radical of formula (I) in position 3 and whose phenyl groups may, apart from position 3, be mono- or poly-substituted by —OH or —O(CH₂)ₙ —H, in which n is from 1 to 8. n preferably denotes 1.

Substitution of the parent substance 2-phenyl-4H-1-benzopyranone by —OH and/or —O(CH₂)ₙ —H occurs preferably in positions 5, 7, 3′, and/or 4′.

Particular preference is given to the use of rutinosides of formula (A):

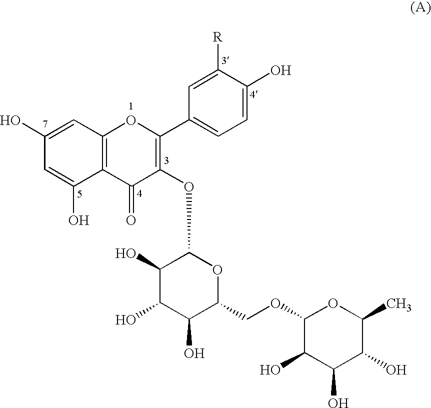

in which R represents H, OH, or OCH₃.

The compound in which R represents H, is known as kaempferol rutinoside; the rutinoside in which R represents OCH₃, is known as isorhamnetin rutinoside. The compound in which R denotes OH, is known as rutin. Accordingly, the process of the invention can produce rhamnose and kaempferol glucoside from kaempferol rutinoside, rhamnose and isoquercetin from rutin, and rhamnose and isorhamnetin glycoside from isorhamnetin rutinoside.

Particularly preferred is the use of the rutinoside rutin.

The starting material used in the process of the invention can be rutinosides in a pure state or alternatively mixtures of rutinosides. The rutinosides may also be contaminated with other flavonoids or with residues from the rutinoside production without the reaction being negatively influenced.

The enzymes used for the enzymatic hydrolysis of the rutinosides can be conventional hydrolases capable of splitting off the rhamnose group from the rutinosides. Use is preferably made of hydrolases obtained from the strain Penicillium decumbens. Particular preference is given to the use of α-L-rhamnosidase as enzymes, as they show a high degree of selectivity toward the hydrolysis of the rhamnose group. Suitable α-L-rhamnosidases are, for example, hesperidinase, naringinase, and those described in Kurosawa et al. (1973), J. Biochem., Vol. 73: 31-37. Use is very preferably made of the enzyme hesperidinase.

Both the rutinosides and the enzymes used in the process of the invention can be procured as commercial products. It is likewise possible to isolate or prepare the starting materials and enzymes by well-known methods.

The enzyme is immobilized on a suitable support. For this purpose use may be made of conventional supports, such as silica gel, for example, commercial spherical or commercial broken silica gels, eg, Lichrosorb®, Lichroprep®, Lichrospher®, and Trisoperl®, and commercial polymeric supports, eg, Eupergit®, Fractogel®, particularly Fractogel epoxy®, and Fractoprep®. Silica gel may be regarded as the preferred support material.

Alternatively, magnetic particles may be used as supports. These are preferably support materials having a magnetic core. This core is usually enveloped by an inorganic oxide. The inorganic oxide is preferably silica gel. Examples of such magnetic supports include MagneSil™ (Promega Corp., Madison, Wis., US), MagPrep™ (Merck) and AGOWAmag™ (AGOWA GmbH, Berlin, Del.). The magnetic supports used may alternatively be magnetic glass particles (eg, MPG (CPG Inc., Lincoln Park, N..J., US)), and also pigments containing magnetite (eg Microna Matte, Mica Black, Colorona Blackstar (all Merck)). Particularly well-suited are nonporous magnetic particles (such as MagPrep) since they cannot give rise to pore obliteration which would lead to drastic deterioration of the enzyme activity.

The enzyme support usually possesses the following properties: the particle size of the support is preferably from 0.005 to 1 mm, and more preferably from 0.01 to 0.5 mm. The pore diameter usually ranges from 10 to 4000 nm, a pore diameter of from 30 to 100 nm being particularly preferred. An adequately large pore size will garantee that the enzyme can be accommodated on the support without loss of activity. The particle surface area is advantageously from 40 to 100m²/g, and the pore volume is preferably selected from a range of from 0.5 to 3 mL/g. In some cases a very large pore diameter of from 2 to 20 μm may be suitable.

The enzyme can be bound by covalent bonds or adsorption. Generally covalent bonding is to be preferred. Examples of a covalent coupling include epoxidation, a carbodiimide method, silanization, a bromocyanogen method, glutaric dialdehyde cross-linking or a dicresyl chloride method (cf Biotransformations and Enzyme Reactions, A. S. Bommarius, Biotechnology (2nd Edition), Vol. 3, pp 427-465, edited by G. Stephanopoulos, V C H Weinheim, Germany 1993, D. R. Walt et al., Trends in Analytical Chemistry, Vol. 13, No. 10, 1994, N. H. Park, H. N. Chang; J. Ferment. Technol., Vol. 57 (4), 310-316, 1979, M. Puri et al.; Enz. Microb. Technol., 18, 281-285, 1996 and H. -Y. Tsen; J. Ferment. Technol., 62 (3), 263-267, 1984). In order to execute this process it is necessary that the support be surface-modified with appropriate functional groups. The functional group can be applied to the support either by copolymerization with functional monomers or by polymer-analogous conversion. Particular preference is given to surface modification with amino groups, aldehyde groups, or epoxide rings, or diol modification. The enzymes can then be covalently bonded to these groups.

The enzymatic hydrolysis takes place in a suitable reactor. A commercial tower is particularly suitable for continuous execution of the process of the invention. When working on a small scale, use can be made, for example, of a tower such as is used for preparative HPLC. The reactor, particularly the tower, should show high hydraulic efficiency. This can be quantified by the number of theoretical plates. It is therefore of advantage to ensure that there is intimate contact of the solution of raw material with the surface of the immobilisate in order to achieve effective utilization of the enzyme and acquire high productivity. The aforementioned preparative HPLC column satisfies these demands and is likewise equipped with appropriate techical means and periphery (pumps, valves, control means). It is also advantageous that detection means, such as UV or RI detection means, have been developed for this purpose so that, if desired, the measurement and control of the degree of conversion achieved by the reaction can be automated.

If magnetic support materials are used for the continuous mode of operation, usually tubular reactors are used which have a contrivance to keep the magnetic particles in stable suspension, eg, electromagnetic coils producing in the flow tube a substantially homogeneous magnetic field whose lines of magnetic flux are parallel to the direction of flow (helmholtz magnetic field). In such magnetically stabilized fluid bed reactors (magnetically stabilized fluidized bed (MSFB)) it is possible to achieve substantially higher flow rates than in conventional fluidized beds or fluid bed columns, which are also suitable for such purposes. This technology can also be used to advantage for catalytic reactions in viscous reaction media.

When the process is to be carried out batchwise, a conventional receptacle, preferably one equipped with an agitator, is suitable. Thus a round-bottomed flask equipped with an agitator can be used on a small scale and a stirred tank on a large scale.

The immobilisate is packed into the reactor prior to the reaction in conventional manner.

The rutinoside to be converted is fed into the reactor, eg, a column or tower, such as a fixed-bed column, usually in the form of a solution or suspension. If the reactor used is a fixed-bed reactor, the rutinoside solution should be completely free from solid material. It is advantageous to predissolve the rutinoside with the solvent in a tank, preferably with stirring and/or heating, in order to achieve optimal solubility. When necessary, prefiltration of the solution can be additionally carried out in order to remove any solid matter. The solvent is preferably an aqueous system in order to guarantee enzyme activity and to prevent possible denaturation. In order to guarantee dissolution of the rutinosides, further solvents may be added. Preferably the process of the invention is carried out in the presence of a solvent mixture of water and at least one organic solvent.

The supplementary organic solvent(s) include both water-miscible and water-immiscible organic solvents.

Suitable solvents for use in the process of the invention are nitrites, such as acetonitrile, amides, such as dimethylformamide, esters, such as acetates, particularly methyl acetate or ethyl acetate, alcohols, such as methanol or ethanol, ethers, such as tetrahydrofuran or methyl-tert-butyl ether, and hydrocarbons, such as toluene.

The process of the invention is preferably carried out in the presence of one or more of the organic solvents ethyl acetate, methanol, ethanol, methyl-tert-butyl ether, or toluene. The process of the invention is very preferably carried out in the presence of one or more acetates, particularly in the presence of methyl acetate, in addition to water.

Suitable ratios of water to organic solvent for the process of the invention are ratios of from 1:99 to 99:1, by volume. The process of the invention is preferably carried out using ratios of water to organic solvent of from 20:80 to 80:20, particularly ratios of from 50:50 to 70:30, by volume.

The amount of rutinoside present in the solvent or solvent mixture in the process of the invention is governed by the solubility of the rutinoside in the solvent or solvent mixture. Optimal execution of the process of the invention is attained when the rutinoside is readily soluble. For this reason it is preferred to operate using a subsaturated solution. Usually the amount of rutinoside in the solvent or solvent mixture is from 0.001 to 5 g/L, preferably from 0.05 to 2 g/L, and more preferably from 0.1 to 1.5 g/L.

The ratio of rutinoside to immobilisate or enzyme depends on the lifetime of the enzyme in the tower or column and its activity in immobilized form.

The reaction is usually carried out at a temperature of from 15° to 80° C. A temperature of from 30° to 60° C. is preferred, and a temperature of from 40° to 50° C. is particularly advantageous for avoiding any possibility of destruction of the enzyme whilst ensuring high solubility of the rutinoside

When the reaction temperature is too low, the reduced enzyme activity causes the reaction to take place at an unduly slow reaction rate. Besides, the solubility of the rutinoside is reduced to such an extent that unnecessarily high amounts of solvent are required. If, on the other hand, the reaction temperature is too high, the enzyme, which is a protein, is denatured and thus deactivated.

When the process of the invention is to be carried out at an elevated temperature, the reactor can be provided with temperature-control means. Common temperature-control means contain a heating coil system or a double jacket. It is furthermore of advantage when the rutinoside to be converted and, in particular, the rutinoside solution, is subjected to temperature control before entry into the reactor. For this purpose, the rutinoside solution is usually withdrawn from a temperature-controlled tank kept at the temperature required for the reaction. Alternatively, the solution to be fed in can be passed through a heated flexible conduit in order to set its temperature to the desired value before entry into the reactor. Said heating can also counteract crystallization of the rutinoside.

Suitable pHs for use in the process of the invention are pHs between 3 and 8. Preferably the process of the invention is carried out at pHs of from 3 to 7, particularly at pHs of from 3 to 6. Furthermore preferred pHs can however vary within the given limits depending on the enzyme used. For example, a pH of from 3.8 to 4.3 is very much preferred when use is made of the enzyme hesperidinase.

Preferably, the process is carried out in such a manner that the pH is adjusted with the aid of a buffer system. Theoretically, all commonly used buffer systems suitable for setting the aforementioned pHs can be employed. Preferably, however, aqueous citrate buffer is used.

The rutinoside mixture, which may be present in the form of a solution or a suspension, is placed in the reactor containing the immobilisate, in order to carry out the enzymatic hydrolysis. This reaction can be carried out continuously or batchwise.

If the reaction is to be carried out batchwise, then a rutinoside suspension is usually placed in the reactor. The degree of conversion is determined by the amounts of rutinoside and immobilisate. Usually, the ratio of rutinoside to immobilisate is from 100:1 to 1:1000, preferably from 10:1 to 1:100, and more preferably from 1:1 to 1:20. The ratio of the immobilisate to the total volume of the suspension is usually from 1:1000 to 1:1, preferably from 1:100 to 1:2, and more preferably from 1:50 to 1:5. The residence time in the reactor normally ranges from 1 h to 10 days, preferably from 8 h to 4 days, and more preferably from 1 to 2 days.

When the reaction is carried out continuously, a rutinoside solution is usually transported steadily through the reactor, preferably a tower or MSFB reactor, by means of a suitable pump. By appropriately setting the flow rate, it is possible to achieve any desired degree of conversion. Normally, the flow rate used is from 0.001 to 1 mm/s, based on the empty tube cross-section of the tower or column.

The activity of the enzyme in the system is found to fall with time. It is therefore necessary to replace the immobilisate at regular intervals either completely or partially. In order to compensate for an activity loss of the enzymes, it is advantageous to evaluate the degree of conversion by UV or RI detection so that when there is a change in composition, this can be counteracted by control via the pump output.

When the reaction solution has left the reactor, the resulting product can be separated. On completion of the reaction, the reaction mixture consists mainly of solvent, unconverted rutinoside, rhamnose, the desired monoglycosidated flavonoid and possibly further additives, such as buffering substances. The monoglycosidated flavonoid usually precipitates when the limit of solubility is reached and gradually accumulates as solid matter.

In the case of a batch operation involving the use of magnetic support materials, the bound enzyme can be separated from the suspension of the product on completion of the reaction in a simple manner with the assistance of a magnetic separating device. On a laboratory scale, a strong permanent magnet in plate form can be used for this purpose. There exist larger separators, however, which have been developed for a great variety of industrial applications and mostly operate on the HGMS principle (high gradient magnetic separation). Such a plant may consist, for example, of a vertical flow tube containing a packing of fine stainless steel wires. Suitably disposed electromagnetic coils produce high magnetic flux gradients along the wires, by which means very efficient separation of even extremely small particles in the order of magnitude of nanometers is achieved. If the magnetic particles are superparamagnetic, lie show no remanent magnetization in the absence of an external magnetic field, they can be readily and completely removed from the separator by repeated rinsing with water after the magnetic field has been switched off.

Isolation of the desired reaction product is carried out by commonly used methods involving conventional workup facilities.

Preferably the product is precipitated by concentration. If the solvent comprises a solvent mixture containing at least one organic solvent, it is preferred that the organic solvent be removed by distillation under reduced pressure. The crystallized monoglycosidated flavonoid is usually separated from the remaining reaction mixture by solid-liquid separation, such as siphoning or filtration under reduced pressure, or by centrifugation of the precipitated crystals. The solid matter is then washed, preferably with water, and then dried.

Alternatively, the entire reactor contents can be first of all filtered off. The filter cake containing the product is then treated with a solvent or a mixture of buffer solvents in which the product is soluble. During this operation the reaction product is extracted from the filter cake.

In batch operation there remains the catalyst, the immobilisate, which is insoluble in this mixture. Is it necessary for the solvent or the mixture of buffer solvents to have no deleterous effect on the enzyme. It has been found that the bound enzyme, eg, naringinase or hesperidinase, possesses in certain buffer solvent mixtures or under mild alkaline conditions no further activity or only a fraction of the original activity but that the activity can be virtually completely recovered if the enzyme is then carefully rinsed with a buffer solution in the pH range of 4-6; thus the activity loss here is only temporary and is not tantamount to irreversible denaturation of the enzyme.

For this procedure, very well-suited extracting agents are tetrahydrofuran buffer mixtures, preferably those having a tetrahydrofuran content of 10-25%, particularly when used at a slightly elevated temperature. Other suitable extracting components are, for example, 1-propanol, 2-propanol, 1,4-dioxane, and methyl acetate. The product can be very readily recovered from the extract by removing the solvent by distillation under reduced pressure and then cooling the aqueous solution containing the product to from 0 ° to 10° C. The reaction product crystallizes from the mother liquor in a state of very high purity.

As an alternative to solvent/buffer mixtures there may be used a dilute ammonia or soda solution as extracting agent, since the reaction product possesses phenolic OH groups which are deprotonated in a weak basic medium; the anion of the reaction product shows comparatively good solubility, but it is also very prone to oxidation, as is noticeable from the gradual discoloration of the extract from yellow to brown. Therefore this variant must be carried out very rapidly, ie the extraction should be completed within a period of from 10 min to 6 h, and preferably from 20 min to 2 h. The operation is preferably carried out under a blanket of protective gas.

In addition, treatment with weakly basic extracting agents, such as aqueous solutions of alkali-metal or ammonium salts of acetic acid, oxalic acid, citric acid, phosphoric acid, boric acid, or carbonic acid, or aqueous solutions of alkylamines, piperidine or pyridine, does not lead to a loss of enzyme activity. The reaction product can be reprecipitated by acidification of the extract and cooling to 0-10° C.

The purity of the resulting monoglycosidated flavonoid when using pure rutinoside is normally greater than 94%. To achieve further purification, the end product may, for example, be recrystallized from suitable solvents, eg from water or solvent mixtures comprising toluene and methanol, or water and methyl acetate.

The solvent remaining after the reaction is preferably recovered in order to maintain the economical value of the process of the invention. Such recirculation is usually carried out continuously and automatically. Available for this purpose are commercial evaporating plants with appropriate control means. If the solvent to be used is a solvent mixture of water and at least one organic solvent, is it not usually possibly to reuse the distillate in the process immediately, since the proportion of solvent is changed by distillation of the organic solvent. By carrying out automatic quality control and correction, it is possible to reestablish the desired proportion of solvent by recirculating the solvent appropriately.

Furthermore concentration can involve membrane processes or nanofiltration. In these processes the solvent mixture is separated without changing its composition.

The following examples are intended to illustrate the present invention. However, they are by no means to be regarded as being restrictive.

### EXAMPLE 1

Immobilization of the enzyme hesperidinase on a silica gel support

1) Conditioning of the Support Surface Prior to Immobilization

1.1) Properties of the Support Material

Silica gel LiChrospher

diameter=15-40 μm

pore diameter=300 Å

particle surface area=80 m²/g

pore volume=0.73 mL/g

density=2 g/mL

1.2) Activation of Silica Gel

250 g of silica gel are mixed with sufficient HCI (7%) in a flask having a capacity of 1 L and allowed to stand overnight in order to moisten the silica gel.

The silica gel suspension is then washed with demineralized water until free from chloride. For this purpose the supernatant liquor must be tested with nitric acid and silver nitrate after each wash. On account of the properties of the silica gel particles, washing is carried out in a ceramic funnel having a diameter of ca 24 \check{s}cm.

1.3) Surface Modification with Amino Groups

In a three-necked flask having a capacity of 2 L and equipped with a reflux condenser and dropping funnel, the acid-treated silica gel is mixed with sufficient water to make it stirrable. At room temperature and with thorough mixing, 1 mmol/g of support comprising 3-aminopropyltrimethoxysilane are added dropwise to the silica gel suspension at a rate of ca 5 drops per second (135 mL of solution being required for 250 g of silica gel). The suspension is then stirred for 2 hours at 90° C. The suspension is then cooled with ice.

The supernatant liquor must be checked for possible residues of 3-aminopropyl-trimethoxysilane by taking pH readings. The suspension of beads is washed with demineralized water until the pH remains constant.

1.4) Coating with Glutaric Dialdehyde

To the resulting silica gel suspension there is added glutaric dialdehyde (GDA) in a concentration of 1 mmol/g of support (13 mL 50% strength GDA solution are required for 250 g of support). The suspension (plus a little water) is rolled in a flask having a capacity of 1 L over a period of 2 hours at room temperature. The suspension is colored yellow at the start, and it is dark red at the end of the procedure.

The supernatant liquor obtained after each wash is checked for residues of glutaric dialdehyde by a precipitation reaction with dinitrophenylhydrazine. The suspension is carefully washed until the test is negative.

2) Immobilization

2.1) Hesperidinase

First Addition of Protein

3.8 g of hesperidinase are stirred in 500 mL of citrate/phosphate buffer mixture (pH 6.0). To improve dissolution, 300 μL of surfactant (Tween 20) are added. The enzyme solution is then filtered.

In a flask having a capacity of 1 L, ca 230 g of the silica gel suspension obtained as described under 1.4) are mixed with the enzyme solution. The suspension of enzyme support is then rolled over a period of ca 40 hours at room temperature.

Second Addition of Protein

ca 0.76 g of hesperidinase (Amano) are stirred in 120 mL of citrate/phosphate buffer mixture (pH 6.0) containing 60 μL of surfactants and subsequently filtered.

The enzyme solution is poured into the aforementioned flask having a capacity of 1 L, and the enzyme solution is rolled at room temperature.

2.2) BSA (for the separation test)

0.3 of Biomex BSA (beef serum albumin powder) are stirred in 100 mL of citrate/phosphate buffer mixture (pH 6.0). In a flask having a capacity of 0.5 L; ca 20 g of the silica gel suspension obtained as described under 1.4) are mixed with the protein solution, and 60 μL of ProClin300 are added.

3) Determination of the Amount and Activity of the Protein

3.1) Amount of Protein (mg of protein per mL)

The protein content of a solution is determined by means of the Bradford test. The standard assay is carried out. This is effected by mixing 20 μL of the sample in 1 mL of Bradford dye reagent (diluted 1:5) and taking a photometric reading at 595 nm after 15 min.

Very small protein concentrations require the use of a microassay. This comprises mixing 0.8 mL of the sample in 0.2 mL of Bradford dye reagent (conc.) and taking a photometrical reading at 595 nm after 15 min.

3.2) Activity

The activity of a solution is measured by reaction thereof with a substitute substrate.

For each sample there are used:

| | |
|---|---|
| 88 μL | of citrate/phosphate buffer mixture (pH = 4.0) |
| 100 μL | of sample |
| 20 μL | of sample |
| 20 μL | of substitute substrate: p-nitrophenyl-α-L-rhamnoside |
| | (rhamnosidase activity) |
| | p-nitrophenyl-α-L-glucoside (glucosidase activity) |

is 1 mL solution is mixed in an Eppendorf reaction vessel. Following an incubation period of 2 min and 5 min repectively at 40° C. in a shaker, every 100 μL of the action mixture are mixed with 1 mL of 1 M soda solution. The concentration of p-nitrophenol is then photometrically measured at 400 nm. The activity is calculated from the concentration change of p-nitrophenol per unit of time.

The activity of an enzyme is given in units (U) (=μmol of converted substrate per minute).

4) Results

| | Amount of Protein | | Activity | | |
|---|---|---|---|---|---|
| | (Standard assay) | | (Substitute substrate) | | |
| | | mg/mL of | | U/mL of | U/mg of pro- |
| Sample¹ | mg | sample | U | sample | tein |

PROTEIN CONTENT and ACTIVITY VALUES
(FREE HESPERIDINASE)

| | | | | | |
|---|---|---|---|---|---|
| Hesp0 | 740 | 1.48 | 48000 | 96 | 65 |
| 1 | 15 | 0.03 | 7 | 0.014 | 0.47 |
| 2 | 20 | 0.04 | 4 | 0.008 | 0.2 |
| Hesp1 | 326 | 2.72 | 11400 | 95 | 35 |
| 3 | 56 | 0.09 | 1450 | 2.33 | 26 |
| 4 | 28 | 0.045 | 361 | 0.58 | 13 |
| 5 | 22 | 0.035 | 95 | 0.15 | 4.3 |
| 6 | 20.5 | 0.033 | 11 | 0.018 | 0.54 |

¹Hesp0: first addition of protein
Hesp1: second addition of protein
Samples 1-6: supernatant liquor

PROTEIN CONTENT and ACTIVITY VALUES
(IMMOBILIZED HESPERIDINASE)

*Protein content of the support ≅ 1,045 g of bound protein
*Located on the support are 4.7 mg of protein/g of support
*2% of the added protein was not bound

### EXAMPLE 2

Production of isoquercetin from rutin by enzymatic hydrolysis using an immobilisate.

In a heated stirred tank having a capacity of 4.5 m³ (1) there are placed 3200 L of demineralized water and 800 L of 1-propanol. The mixture is heated to ca 50-60° C. via the steam inlet (2). 8000 g of rutin, DAB are added to the solution with vigorous stirring. The mixture is stirred until the rutin has completely dissolved. The pH is then monitored via a circulating pump and an in-line pH meter (3a) and set to pH to 4.0-4.5 (using H₃ PO₄ and NaOH) when necessary. A sample may be taken via the manual valve (4) for inspection purposes and determination of the concentration.

To start the reaction, the solution is fed through a bag filter (5) and a tube filter (6) to a piston-type dosing pump (7). The bag filter is responsible for stopping the major amount of undissolved components, while the tube filter cleans the solution to a degree of fineness of 0.2 μm.

The piston-type dosing pump (7) transports the solution through a heatable flexible tube, which adjusts the temperature of the solution via a thermometer to 40 ° C. at the input of the column, the rate of flow to the column (9) (100×400 mm) being 1 L/min. The column contains 1.5 kg of immobilisate. Since the electrically heated flexible tube cannot cool the solution, the temperature in the stirred tank (1) is thus set to such a value that cooling occurring en route to the pump gives, at maximum pump delivery, a temperature of up to 40° C.

A sample can be taken from the solution after percolation through the column, via a manual valve (10), by means of which the temperature and the degree of conversion attained by the reaction can be measured off-line. If the measured degree of conversion is lower than required, the output of the pump is reduced appropriately.

The reaction is completely finished when the solution has passed through the column so that the solution can be passed on to the collection vessel (11). There the solution is reduced in volume by ca 10-20% via a condenser (12). By this means the content of propanol is reduced considerably, which means that the solubility of the isoquercetin drops steeply. Subsequent cooling causes the solubility to drop further so that the product precipitates and can be separated in a bag filter (13). From here it is passed to a drying oven (14) for desiccation. The mother liquor and the distilled condensate are together recycled for reuse in the stirred tank (1).

### EXAMPLE 3

1. Modification of Silica Gel Particles with Aldehyde Groups and Immobilization of Naringinase on this Particles

400 mL of 10% strength HCI were poured over 250 g of silica gel (eg LiChrospher Si 300, Merck, Darmstadt) in a vessel capable of being sealed, after which the vessel was degassed for 10 min by supersonics and left to stand for a period of 24 h at room temperature. The silica gel was then filtered off and washed with several liters of demineralized water until the pH was >4.5 and no more chloride ions could be detected in the filtrate (spot reaction with a solution of AgNO₃ in acetic acid).

The acid-treated moist silica gel was placed in a three-necked flask having a capacity of 4 L and equipped with a precision glass stirrer, a reflux condenser and a 100 mL dropping funnel and was slurried therein with 3 L of demineralized water. 100 mL of aminopropyltrimethoxysilane (ABCR, Karlsruhe) were added through the dropping funnel with stirring over a period of 15 min. The suspension was then heated and stirred at 90° C. for 90 min. The cooled suspension was filtered and washed eight times with 1 L of demineralized water each time.

The aminoactivated silica gel was suspended in 3 L of water, which had been degassed by supersonics, in a three-necked flask having a capacity of 4 L and equipped with a precision glass stirrer and 100 mL dropping funnel; the pH was lowered to 8.0 with a few drops of 2 M acetic acid. 100 mL of 50% strength glutaric dialdehyde solution (Merck, Darmstadt) were then added dropwise over a period of 1 h, and the suspension was stirred for a further 2.5 h at room temperature. The activated silica gel was refiltered and washed with ice-cold demineralized water until no more glutaraldehyde could be detected in the wash water (spot reaction with a solution of 2,4-dinitrophenylhydrazine in sulfuric acid).

The silica gel modified with aldedyde groups was suspended in 500 mL of demineralized water in a flask having a capacity of 4 L by agitation with a precision glass stirrer. 13 g of naringinase (Sigma, Deisenhofen) were dissolved in 2.5 L of 0.25 M phosphate buffer, pH 8.0. The enzyme solution was added to the silica gel suspension and stirred for 96 h at room temperature. The immobilisate was then filtered off and washed a number of times first of all with 0.2 M sodium chloride solution and then with 50 mM citrate buffer, pH 4.0. The rhamnosidase activity of the immobilisate was determined with p-nitrophenyl-L-α-rhamnopyranoside (Sigma, Deisenhofen) as substrate by the Kurosawa method; it was 120 U/g.

2. Immobilization of Hesperidinase on Eupergit™ C

50 g of Eupergit (Röhm, Weiterstadt) were mixed with 300 mL of 0.8 M potassium phosphate buffer, pH 8.5, in a 500 mL glass bottle having a screw lid, and allowed to stand for 30 min. 5.0 g of hesperidinase (Amano) were then added and the batch was agitated for 120 h at room temperature on a rolling mixer. The Eupergit was filtered off by means of a sintered-glass filter and washed a number of times first with 0.2 M sodium chloride solution, then twice with 1 L of 0.1 M citrate buffer, pH 4.0, each time. The rhamnosidase activity of the immobilisate was determined by the Kurosawa method using p-nitrophenyl-L-α-rhamnopyranoside (Sigma, Deisen-hofen) as substrate; it was 15 U/g, based on dry immobilisate, or 4.2 U/g, based on moist immobilisate.

3. Conversion of Rutin to Isoguercetin using Hesperidinase which has been Immobilized on Eupergit in a Stirred-tank Reactor followed by Extraction of the Product with a Tetrahydrofuran/buffer Mixture

In a round-bottomed flask having a capacity of 2000 mL there were stirred together 1000 mL of 50 mM citrate buffer, pH 4.0, 100 g (wet weight) of naringinase immobilized on Eupergit and having an activity of 4.2 U/g, and 10 g of rutin (Merck, Darmstadt) at 40° C. using a precision glass stirrer; the degree of conversion was continuously determined by HPLC analysis. After a total of 96 h, the reactor contents were filtered off through a Büchner filter. The filter cake was returned to the round-bottomed flask and stirred at 40° C. for 30 min in a mixture of 400 mL of 50 mM citrate buffer, pH 4.0, and 100 mL of tetrahydrofuran, during which operation the major portion of the isoquercetin dissolved. The mixture was filtered hot and the filter cake re-extracted with 500 mL of buffer/tetrahydrofuran mixture for 30 min. Following filtration, the two isoquercetin extracts were combined with the first filtrate, and the tetrahydrofuran was removed with the aid of a rotation evaporator. In order to precipitate the product completely, the aqueous isoquercetin solution was cooled to 4° C. Following filtration and drying in a desiccator a yield of 5.8 g of product was obtained, which comprised of 98% isoquercetin and 2% rutin.

The moist Eupergit was washed once with cold tetrahydrofuran buffer mixture, then repeatedly with 50 mM citrate buffer, pH 4.0, until the smell of tetrahydrofuran was only weakly discernible. The activity of the enzyme was still 3.6 U/g, which is equivalent to an activity loss of 14%.

4. Conversion of Rutin to Isoguercetin with Hesperidinase that has been Immobilized on Eupergit in a Stirred-tank Reactor followed by Extraction of the Product with an Alkaline Buffer Solution

In a round-bottomed flask having a capacity of 2000 mL there were stirred together 1000 mL of 50 mM citrate buffer, pH 4.0, 100 g of naringinase that had been immobilized on Eupergit and had an activity of 4.2 U/g (wet weight), and 10 g of rutin (Merck, Darmstadt) at 40° C. using a precision glass stirrer; the degree of conversion was continuously determined by HPLC analysis. After a total of 96 h the reactor contents were filtered off through a Büchner filter. The wet cake was returned to the round-bottomed flask and stirred in 300 mL of 50 mM sodium carbonate buffer, pH 10.0, at room temperature for 5 min, during which operation a portion of the isoquercetin dissolved to give an intensely yellow color. The suspension was filtered and the filter cake immediately re-extracted with carbonate buffer. After a total of 7 extraction cycles the Eupergit was substantially free from color and the isoquercetin was virtually completely dissolved. The extracts were combine, carefully acidified with dilute hydrochloric acid until the pH was approximately 3, and the mixture was then cooled to 4° C. Following filtration and drying in a desiccator a yield of 4.9 g of product was obtained, which comprised 98% isoquercetin and 2% rutin.

The moist Eupergit was washed twice with 50 mM citrate buffer and was then again ready for use for further reactions. The activity of the enzyme was still 3.9 U/g, which is equivalent to an activity loss of 7%.

### EXAMPLE 4

1. Modification of Magnetic Silica Particles with Aldehyde Groups and Immobilization of Naringinase on this Particles

In a three-necked flask having a capacity of 1 L and equipped with precision glass stirrer, dropping funnel and reflux condenser, there was placed a suspension of 30 g of magnetic silica particles (MagPrep, Merck, Darmstadt) in 600 mL of water. A mixture of 20 mL of aminopropyltriethoxysilane (ABCR, Karlsruhe) and 20 mL of isopropanol was added dropwise over a period of 30 min with stirring. The mixture was then heated to 85° C. and stirred for 1 h at this temperature. Following cooling, the suspension was placed in a beaker, the particles were collected at the bottom of the vessel by means of a strong permanent magnet, and the supernatant liquor was decanted. The particles were repeatedly washed with demineralized water until the pH of the washings remained constant. The particles were then resuspended in 600 mL of water, and the pH was adjusted to a value of ca 8 with a few drops of acetic acid; following the addition of 24 mL of 50% strength glutaric dialdehyde solution, the suspension was stirred for 4 h at room temperature and the particles were then washed with demineralized water until no more glutaraldehyde could be detected in the washings (spot reaction with 2,4-dinitrophenylhydrazine solution in sulfuric acid).

The aldehyde-derived particles were resuspended in 600 mL of 0.2 M potassium phosphate buffer, pH 9, in a round-bottomed flask having a capacity of 1 L. Following the addition of a solution of 1 g of naringinase (Sigma, Deisenhofen) in 100 mL of 50 mM sodium chloride solution, the mixture was stirred with a precision glass stirrer over a period of 2 days at room temperature. The particles were then separated with the aid of a permanent magnet and repeatedly washed first of all with 0.2 M sodium chloride solution and then with 50 mM citrate buffer, pH 4.0. The rhamnosidase activity of the immobilisate was determined by the Kurosawa method using p-nitrophenyl-L-α-rhamnopyranoside (Sigma, Deisenhofen) as substrate (Kurosawa, Ikeda, Egami, J. Biochem. 73, 31-37 (1973): α-L-rhamnosidase of the liver of Turbo cornutus and Aspergillus niger); it was 162 U/g.

2. Modification of Magnetic Silica Particles with Epoxide Rings and Immobilization of Naringinase on this Particles

In a three-necked flask having a capacity of 1 L and equipped with precision glass stirrer, dropping funnel, and reflux condenser, there was placed a suspension of 30 g of magnetic silica particles (MagPrep, Merck, Darmstadt) in 600 mL of 50 mM sodium acetate solution. A mixture of 20 mL of (3-glycidoxypropyl)trimethoxysilane (ABCR, Karlsruhe) and 20 mL of isopropanol was added dropwise over a period of 30 min with stirring, and the mixture was then heated to 85° C. and stirred at this temperature for 1 h. Following cooling, the suspension was placed in a beaker, the particles were collected at the bottom of the vessel by means of a strong permanent magnet and the supematant liquor was decanted. The particles were repeatedly washed with demineralized water until the pH of the washings remained constant. In order to quantitate the epoxide rings, a sample of ca 0.5 g of the material was repeatedly washed with methanol and then dried to constant weight at ca 70° C. in a drying oven. Determination of the epoxide rings was carried out by the Pribyl method (Pribyl, Fresenius Z Anal. Chem. 303, 113-116 (1980): Bestimmung of Epoxydendgruppen in modifizierten chromatographischen Sorbentien and Gelen) gave a value of 250 μmol/g.

150 mL of a 20% strength (w/v) suspension of epoxy-derived magnetic particles were mixed with 350 mL of 1 M potassium phosphate buffer, pH 9.0, in a round-bottomed flask having a capacity of 1 L. Following the addition of a solution of 1.5 g of naringinase (Sigma, Deisenhofen) in 15 mL of 50 mM sodium chloride solution, the mixture was stirred with a precision glass stirrer for 16 h at 40° C. The particles were then separated with the aid of a permanent magnet and repeatedly washed first of all with 0.2 M sodium chloride solution and then with 50 mM citrate buffer, pH 4.0; The rhamnosidase activity of the immobilisate was determined with p-nitrophenyl-L-α-rhamnopyranoside (Sigma, Deisenhofen) as substrate by the Kurosawa method; it was 102 U/g.

3. Modification of Magnetic Silica Particles with Carboxyl Groups and Immobilization of Naringinase on this Particles

In a three-necked flask having a capacity of 1 L and equipped with precision glass stirrer, dropping funnel and reflux condenser, there was placed a suspension of 30 g of magnetic silica particles (MagPrep, Merck, Darmstadt) in 600 mL of water. A mixture of 28 mL of 3-(triethoxysilyl)propylsuccinyl anhydride (ABCR, Karlsruhe) and 28 mL of isopropanol was added dropwise over a period of 30 min with stirring, and the pH of the reaction mixture then adjusted to 9.0 by dropwise addition of a 10% strength sodium hydroxide solution. The mixture was heated to 80° C. and stirred for 2 h at this temperature. During this operation, the pH was controlled at regular intervals and corrected, if need be, by the addition of alkali. Following cooling, the suspension was placed in a beaker, and the particles were collected at the bottom of the vessel by means of a strong permanent magnet, and the supematant liquor was decanted. The particles were washed three times with demineralized water, once with a 2 M acetic acid solution and then repeatedly with demineralized water until the pH of the washings remained constant.

150 mL of a 20% strength (w/v) suspension of carboxylderived magnetic particles were mixed with 300 mL of 0.4 M potassium phosphate buffer, pH 5.0, and a solution of 1.5 g of naringinase (Sigma, Deisenhofen) in 150 mL of 50 mM sodium chloride solution in a round-bottomed flask having a capacity of 1 L. Following the addition of 8 mL of a 1% strength (w/v) solution of EDC (N -ethyl-N′-(3-dimethylamino-propyl)carbodiimide hydrochloride, Merck, Darmstadt) in water, the mixture was stirred for 20 h at room temperature. The particles were then separated with the aid of a permanent magnet and repeatedly washed first of all with 0.2 M sodium chloride solution and then with 50 mM citrate buffer, pH 4.0. The rhamnosidase activity. of the immobilisate was determined with p-nitrophenyl-L-α-rhamnopyranoside (Sigma, Deisenhofen) as substrate by the Kurosawa method; it was 71 U/g.

4. Modification of Magnetic Mica Pigments with Aldehyde Groups and Immobilization of Hesperidinase on these Particles

In a three-necked flask having a capacity of 1 L and equipped with a precision glass stirrer, dropping funnel, and reflux condenser there was placed a suspension of 30 g of magnetic mica pigments (“Colorona Blackstar Green”, Merck, Darmstadt) in 300 mL of water. A mixture of 20 mL of aminopropyltriethoxysilane (ABCR, Karlsruhe) and 20 mL of isopropanol was added dropwise over a period of 30 min with stirring. The mixture was then heated to 85° C. and stirred for 1 h at this temperature. Following cooling, the suspension was placed in a beaker, the particles were collected at the bottom of the vessel by means of a permanent magnet, and the supematant liquor was decanted. The particles were repeatedly washed with demineralized water until the pH of the washings remained constant. The particles were then resuspended in 300 mL of water and the pH was adjusted to a value of ca 8 with a few drops of acetic acid; following the addition of 25 mL of 50% strength glutaric dialdehyde solution, the suspension was stirred for 4 h at room temperature and the particles were then washed with demineralized water until no more glutaraldehyde could be detected in the wash water (spot reaction with 2,4-dinitrophenylhydrazine solution in sulfuric acid).

30 g of aldehyde-derived mica pigments “Colorona Blackstar Green” were resuspended in 300 mL of 0.2 M potassium phosphate buffer, pH 7.5, in a round-bottomed flask having a capacity of 1 L. Following the addition of a solution of 1 g of hesperidinase (Amano) in 20 mL of 0.2 M potassium phosphate buffer, pH 7.5, the mixture was stirred with a precision glass stirrer over a period of 3 days at room temperature. The particles were then separated with the aid of a permanent magnet and repeatedly washed first of all with 0.2 M sodium chloride solution and then with 50 mM citrate buffer, pH 4.0. The rhamnosidase activity of the immobilisate was determined using p-nitrophenyl-L-α-rhamnopyranoside (Sigma, Deisenhofen) as substrate by the Kurosawa method; it was 10 U/g.

5. Conversion of Rutin to Isoguercetin with Immobilized Naringinase in a Stirred-tank Reactor and Isolation of the Magnetic Biocatalyst with a Permanent Magnet

In a double-walled stirred reactor having a capacity of 500 mL there were stirred together 400 mL of 50 mM citrate buffer, pH 5.0, 20 g of naringinase immobilized on magnetic silica particles and having an activity of 102 U/g, and 10 g of rutin (Merck, Darmstadt) at 40° C.; the degree of conversion was determined at intervals by HPLC analysis. Following a period of 24 h, the reactor contents were pumped into a beaker and the catalyst was collected at the bottom of the vessel with the aid of a plate magnet (Bakker, 200 mT). The supernatant liquor was immediately filtered off in vacuo with the aid of a pump, and the magnetic particles were washed a number of times with 100 mL of buffer each time in order to rinse off the final residues of adhesive solid isoquercetin. The collected isoquercetin was filtered off, washed a number of times with small amounts of ice water and dried in a desiccator. The yield was 6.5 g. HPLC analysis gave a composition of 96% isoquercetin, 2% quercetin, and 2% rutin. The activity of the immobilisate following conversion was still 92 U/g. This is equivalent to an activity loss of 10%.

6. Conversion of Rutin to Isoguercetin with Immobilized Naringinase in a Stirred-tank Reactor and Separation of the Magnetic Biocatalyst with an Electromagnetic Separator (FIG. 1)

In a double-walled stirred reactor having a capacity of 500 mL there were stirred together 300 mL of 50 mM citrate buffer, pH 5.0, 10 g of naringinase immobilized on magnetic silica particles and having an activity of 102 U/g, and 5 g of rutin (Merck, Darmstadt) at 40° C.; the degree of conversion was continuously determined by HPLC analysis. Following a period of 24 h, the reactor contents were passed through an electromagnetic HGMS plant with the aid of a peristaltic pump producing a flow rate of 25 mL/min, by which means the magnetic particles were completely separated onto the wire matrix (technical data on the separating plant: glass pipe having an inside diameter of 20 mm and a length of 200 mm, capacity 65 mL, weight of the wire packing of SS alloy 15 g, 4 series-connected coils, current strength 6 A, magnetic field strength of the Helmholtz field 25 mT). The magnet field was activated and citrate buffer was pumped twice through the column in an amount of 100 mL each time, in order to rinse off the magnetic particles. The combined suspensions of the product were filtered, and the isoquercetin was washed with ice water and dried in a desiccator. The yield was 3.1 g. HPLC analysis gave a composition of 97% isoquercetin, 2% quercetin, and 1% rutin.

To reclaim the catalyst, the magnetic field was deactivated and 100 mL of citrate buffer, pH 5.0, were pump-circulated through the separating plant for 10 min at a flow rate of 100 mL/min, the direction of flow being changed a number of times. The catalyst suspension was then pumped back into the stirred tank and the remaining amount of catalyst still in the precipitator was again rinsed off twice with 100 mL of citrate buffer each time. The activity of the immobilisate following conversion was still 94 U/g. This is equivalent to an activity loss of 8%.

7. Conversion of Rutin to Isoqureretin with Hesperidinase Immobilized on Mica Particles in a Stirred-tank Reactor and Isolation of the Magnetic Biocatalyst using a Plate Magnet

In a double-walled stirred reactor having a capacity of 500 mL there were stirred together 400 mL of 50 mM citrate buffer, pH 5.0, 30 g of hesperidinase immobilized on “Colorona Blackstar” and having an activity of 10 U/g and 5 g of rutin (Merck, Darmstadt) at 40° C.; the degree of conversion was continuously determined by HPLC analysis. Following a period of 24 h, the reactor contents were pumped into a beaker and the catalyst was collected at the bottom of the vessel with the aid of a plate magnet (Bakker, 200 mT). The supernatant liquor was immediately filtered off in vacuo with the aid of a pump, and the magnetic particles were washed a number of times with 100 mL of buffer each time in order to rinse off the final residues of adhering solid isoquercetin. The collected isoquercetin was filtered off, washed a number of times with small amounts of ice water and dried in a desiccator. The yield was 3.3 g. HPLC analysis gave a composition of 96% isoquercetin and 4% rutin. The activity of the immobilisate following conversion was still 9.7 U/g. This is equivalent to an activity loss of 3%

8. Conversion of Rutin to Isoguercetin with Naringinase Immobilized on Magnetic Silica Gel Particles in an MSFB Reactor

In a receiving flask there was stirred a mixture of 5 g of rutin, 900 mL of 50 mM citrate buffer, pH 5.0, and 100 mL of methyl acetate at 40° C. until an approximately homogeneous suspension containing no conspicuous agglomerates resulted. The reactor was equipped for temperature control. The methyl acetate was added in order to increase the solubility and the redissolving rate at room temperature, and to prevent the formation of quercetin. In the meantime a suspension of 6 g naringinase immobilized on magnetic silica particles and having an activity of 162 U/g in 60 mL of 50 mM citrate buffer, pH 5.0, was pumped into the tube of the MSFB reactor with the magnetic field deactivated. A magnetic field was set to 20 mT and fresh citrate buffer was first of all introduced upwardly with the aid of a piston pump designed for precision metering at a rate of flow of 5 mL/min until the particles had reached a stable state in the magnetic field. The rutin suspension was then pumped through the MSFB reactor at room temperature over a period of 3.5 h. The methyl acetate was first of all removed from the mixture of products in a rotary film evaporator and the product was then filtered off, washed a number of times with ice water, and dried in a desiccator. The product yield was 2.9 g; the product comprised 86% isoquercetin and 14% rutin.

1. A process for the production of a monoglycosidated flavonoid by enzymatic hydrolysis of a rutinoside of formula (A) 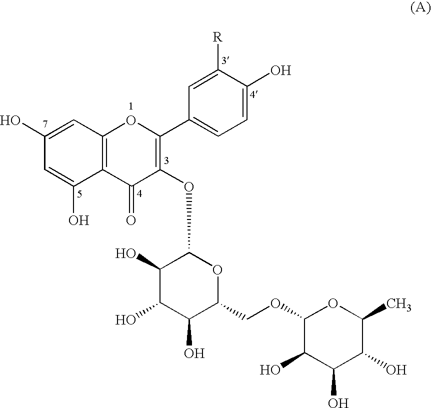
in which R denotes H, OH, or OCH₃, wherein said enzymatic hydrolysis is carried out using an enzyme immobilized on a support.

2. A process as defined in claim 1, wherein the rutinoside used is rutin.

3. A process as defined in claim 1 or claim 2, wherein the enzyme used is an (α-L-rhamnosidase.

4. A process as defined in any one of claims 1 to 3, wherein the enzyme used is hesperidinase.

5. A process as defined in any one of claims 1 to 4, wherein the enzyme is immobilized on silica gel.

6. A process as defined in any one of claims 1 to 4, wherein the enzymatic hydrolysis is carried out in the presence of a solvent mixture of water and at least one organic solvent.

7. A process as defined in any one of claims 1 to 6, wherein the reaction is carried out at a reaction temperature of from 15° to 80° C.

8. A process as defined in any one of claims 1 to 7, wherein the reaction is carried out at a pH of from 3 to 8.
